SEQUENCE LISTING (3 sequences)

SEQ ID NO: 1 - DNA, 24 bp, Homo sapiens
gctctggccttctggtatgggacc

SEQ ID NO: 2 - DNA, 24 bp, Homo sapiens
gctctggccttctggtatgggacc

SEQ ID NO: 3 - DNA, 24 bp, Homo sapiens
gctctggccttctggtatgggacc

(19) United States
(12) Patent Application Publication
(10) Pub. No.: US 2009/0099197 A1
(43) Pub. Date: Apr. 16, 2009

(54) Methods of Identifying and Treating Individuals Exhibiting MDR-1 Overexpression With Protein Tyrosine Kinase Inhibitors and Combinations Thereof

(75) Inventor: Francis Y. Lee, Yardley, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/084,861

(22) PCT Filed: Nov. 14, 2006
(86) PCT No.: PCT/US2006/044214

§ 371 Date: Dec. 17, 2008

Related U.S. Application Data
(60) Provisional application No. 60/736,671, filed on Nov. 15, 2005.
(60) Provisional application No. 60/838,455, filed on Aug. 17, 2006.

(51) Int. Cl. A61K 31/497; G01N 33/53; A61P 35/00

(52) U.S. Cl. 514/252.19; 435/7.1

(57) ABSTRACT

The invention described herein relates to diagnostic and treatment methods and compositions useful in the management of disorders, for example cancers, involving cells that overexpress MDR-1, and methods of decreasing the incidence of CNS complications that are often associated with CML patients that have been administered imatinib or other protein tyrosine kinase inhibitors.

This application claims benefit to provisional application U.S. Ser. No. 60/736,671 filed Nov. 15, 2005; and to provisional application U.S. Ser. No. 60/838,455, filed Aug. 17, 2006; under 35 U.S.C. 119(e). The entire teachings of the referenced applications are incorporated herein by reference.

### FIELD OF THE INVENTION

The invention described herein relates to diagnostic and treatment methods and compositions useful in the management of disorders, for example cancers, involving cells that overexpress MDR-1, and methods of decreasing the incidence of CNS complications that are often associated with CML patients that have been administered imatinib or other protein tyrosine kinase inhibitors.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise and are predicted to become the leading cause of death in the developed world.

BCR-ABL, a fusion oncogene generated by a reciprocal translocation between Chromosomes 9 and 12, encodes the BCR-ABL fusion protein, a constitutively active cytoplasmic tyrosine kinase present in >90% of all patients with chronic myelogenous leukemia (CML), and in 15-30% of adult patients with acute lymphoblastic leukemia (ALL).¹⁻³ Numerous studies have demonstrated that the underlying pathophysiology of CML is the kinase activity of BCR-ABL.^{1,4} The clinical success of the BCR-ABL kinase inhibitor imatinib (Gleevec®) has validated its use in the management of CML. Imatinib is particularly effective in the early (chronic) phase of the disease, where the complete hematologic response (CHR) rate can be in excess of 90%.^{5,6} However, patients with advanced disease (accelerated phase and blast crisis) and Philadelphia chromosome positive ALL (Ph+ALL) have been less sensitive to imatinib. Furthermore, responses are transient, generally lasting less than 6 months,⁷⁻¹⁰ and, clinical resistance to imatinib, both innate and acquired, has been observed in all phases of disease,^{11,12} which may limit treatment benefits of imatinib in the long term.

Multiple mechanisms underlie imatinib resistance, including: BCR-ABL gene mutations;¹³ overexpression of BCR-ABL or amplification of the BCR-ABL gene locus;¹³ activation of BCR-ABL-independent pathways, such as members of the SRC kinase family;¹⁴ binding to serum α-1 acid glycoprotein;¹⁵ and increased drug efflux (via MDR-1).^{16,17}

The use of combinations of BCR-ABL and SRC inhibitors, or multi-targeted inhibitors of both kinases, to address these problems has been suggested and, in some cases, assessed preclinically.¹⁸⁻²¹ Dasatinib (N-[2-Chloro-6-methylphenyl]-2-[6-[4-[2-hydroxyethyl]piperazin-1-yl]-2-methylpyrimidin-4-ylamino]thiazole-5-carboxamide; formerly N-(2-chloro-6-methylphenyl)-2-[[6-[4-(2-hydroxyethyl)-1-piperazinyl]-2-methyl-4-pyrimidinyl]amino]-5-thiazolecarboxamide) is a novel, potent, orally available, multi-targeted kinase inhibitor, which combines nanomolar inhibition of BCR-ABL kinase activity with similar potency against members of the SRC family of cytoplasmic tyrosine kinases. The synthesis and biochemical properties of this compound have been presented previously.²² The ability of dasatinib to show potent activity against 18 of 19 common mutations in BCR-ABL that are known to confer imatinib resistance has been demonstrated preclinically, both in vitro and in vivo.²³⁻²⁴ Furthermore, the results of a Phase I dose finding study support these preclinical data in the clinical setting.^{25,26}

In light of the limitations of imatinib therapy in CML, and the lack of therapeutic options for patients who are refractory to, or intolerant of, imatinib treatment, a clear medical need exists for more effective therapeutic options, particularly in advanced disease and Ph+ALL. The invention provided herein satisfies this need.

### SUMMARY OF THE INVENTION

The present invention provides a method of screening a biological sample, for example cells that do not respond, or that have stopped responding, or that have a diminished response, to kinase inhibitors used to inhibit proliferation of said cells. For example, the present invention provides a method of screening cells from an individual suffering from cancer who is either being treated with imatinib or is imatinib naive, and whose cells do not respond or have stopped responding or that have a diminished response to imatinib, for overexpression of MDR-1 relative to a standard. If MDR-1 overexpression is present, administration of a therapeutically acceptable amount of dasatinib, alone or in combination with imatinib or another protein tyrosine kinase inhibitor, is warranted to inhibit proliferation of said cells. Wherein said cancer is CML.

The present invention provides a method of diminishing the incidence of CNS complications for CML patients, said method comprising the step of screening a biological sample to identify cells, that do not respond, or that have stopped responding, or that have a diminished response, to protein tyrosine kinase inhibitors used to inhibit proliferation of said cells. For example, the present invention provides a method of screening cells from an individual suffering from cancer who is either being treated with imatinib or who is imatinib naive, and whose cells do not respond or have stopped responding or that have a diminished response to imatinib, for overexpression of MDR-1 relative to a standard. If MDR-1 overexpression is present, administration of a therapeutically acceptable amount of dasatinib, alone or in combination with imatinib; another protein tyrosine kinase inhibitor; a farnysyl transferase inhibitor (e.g., Compound II); a tubulin stabilizing agent (e.g., pacitaxol, epothilone, taxane, etc.); an increased dosing frequency of dasatinib; a more aggressive dosing regimen of dasatinib; or an increased dose of dasatinib; is warranted to inhibit proliferation of said cells. Said method of identifying patients with increased MDR-1 expression will also lead to diminished incidence of CNS complications in susceptible patients due to the ability of dasatinib to overcome MDR-1 overexpression resistance which is a common complication in CNS patients administered imatinib due to the significantly decreased sensitivity of MDR-1 overexpressing cells to imatinib. Wherein said cancer is CML.

The present invention also provides a method of identifying patients by screening a sample to identify cells that overexpress MDR-1 relative to a standard level, whereby the presence of MDR-1 overexpression indicates that increased levels of a therapeutically acceptable amount of dasatinib is warranted to inhibit proliferation of said cells.

The present invention also provides a method of screening a sample to identify cells that overexpress MDR-1 relative to a standard level, whereby the presence of MDR-1 overexpression indicates that increased levels, more aggressive dosing regimen, or increased dosing frequency, of a therapeutically acceptable amount of dasatinib is warranted to inhibit proliferation of said cells, wherein said increased level is 10, 20, 30, 40, 50, 60, 70, 80, 90, or 95% more than the prescribed dasatinib dose, or 1.5×, 2×, 2.5×, 3×, 3.5×, 4×, 4.5×, or 5× more dasatinib than the prescribed dose.

Said administration of dasatinib may be either alone or a combination with imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor, especially a BCR-ABL inhibitor such as imatinib as indicated herein, or AMN107; an increased dosing frequency of N-(2-chloro-6-methylphenyl)-2-[[6-[4-(2-hydroxyethyl)-1-piperazinyl]-2-methyl-4-pyrimidinyl]amino]-5-thiazolecarboxamide; a higher dose of dasatinib; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. Additional combinations comprising N-(2-chloro-6-methylphenyl)-2-[[6-[4-(2-hydroxyethyl)-1-piperazinyl]-2-methyl-4-pyrimidinyl]amino]-5-thiazolecarboxamide that may be useful to practice the methods of the present invention are described in U.S. Ser. No. 10/886,955, filed Jul. 8, 2004, U.S. Ser. No. 60/632,122, filed Dec. 1, 2004, and U.S. Ser. No. 60/678,030, filed May 5, 2005, each of which are incorporated herein by reference.

### BRIEF DESCRIPTION OF THE FIGURES

FIGS. 1A-B shows that imatinib resistant (K562-R) cells are sensitive to dasatinib and that imatinib resistant cells show only a modest increase in IC50. Kidney K562 cells and imatinib resistant cells were incubated with either dasatinib (▴); imatinib (⋄); or AMN107 (♦) and the percent growth inhibition was measured as described in Example 1. As shown in (A), K562 cells were significantly more sensitive to dasatinib than either imatinib or AMN107 alone. In addition, imatinib resistant cells (K-562R) exhibited pronounced resistance to imatinib requiring at least 56 fold more imatinib to achieve 50% inhibition than the imatinib-sensitive cells. In contrast (B), imatinib-resistant cells were only modestly less sensitive to dasatinib than the imatinib-sensitive cells requiring 6 fold higher levels of dasatinib to achieve 50% inhibition. Experiments were performed as described in Examples 1 and 2.

FIGS. 2A-B. FIG. 2A shows K562/ADM cells highly express MDR as shown using FACS with an 4E3 antibody selective for P-gp. Normal K562 cells are shown on the left, while the MDR-overexpressing K562/ADM cells are shown on the right. FIG. 2B shows the in vitro antitumor activity of dasatinib and adriamycin against imatinib-resistant (K562R) and imatinib-resistant MDR-1 overexpressing cells (K562/ADM). While K562/ADM cells are 60 fold more resistant to adriamycin relative to normal K562 cells, they are only 6 fold more resistant to dasatinib. Experiments were performed as described in Examples 1 and 2, and MDR-1 expression was performed as described in Example 5.

FIGS. 3A-B shows the in vitro antitumor activity of dasatinib against imatinib-resistant and imatinib-resistant MDR-1 overexpressing cells shown in FIGS. 2A-B is equally effective in human in vivo CML models in SCID mice. (A) Activity versus imatinib-resistant K562/STI-571/R tumor xenografts. Δ=50 mg/kg dasatinib; ▴=5 mg/kg dasatinib; ♦=150 mg/kg imatinib; ⋄=75 mg/kg imatinib; •=control. (B) Activity versus K562/ADM tumor xenografts. Mice were implanted subcutaneously (SC) on Day 0 with K562/ADM. On Day 18 when tumors were at a size range of 100-200 mg, treatment began. Dasatinib was administered PO, QD×14, at doses of 5, 15 and 30 mg/kg. Each symbol represents the median tumor weight of a group of six mice. ▴=30 mg/kg dasatinib; ⋄=15 mg/kg dasatinib; ▪=5 mg/kg dasatinib; •=control. Experiments were performed as described in Example 3.

FIGS. 4A-B shows dasatinib prolongs the survival of mice bearing intracranial K562 CML leukaemia. As shown in FIG. 4A, 5 mg/kg dasatinib increased survival 268% relative to control, whereas 15 mg/kg dasatinib increased survival by 450% relative to control. Concentrations and dosing frequency are as specified in FIG. 4A. As shown in FIG. 4B, brain penetration of dasatinib was about 5-10% of the concentration observed in blood plasma, but this was sufficient to kill K562 cells and maintain efficacy due to sub-nM potency of dasatinib. Experiments were performed as described in Example 4.

FIGS. 5A-B shows dasatinib administration results in significant reduction in intracranial K562 cells in SCID mice using spectrophotometric bioluminescence. As shown in FIG. 5A, control mice show significant bioluminescence, while marked reduction of bioluminescence is observed after treating mice with dasatinib (15 mg/kg, 2qdx14;6,M-F,po). Experiments were performed as described in Example 4.

### DETAILED DESCRIPTION OF THE INVENTION

Imatinib is a small-molecule inhibitor of the BCR/ABL tyrosine kinase that produces clinical remissions in CML patients with minimal toxicity relative to older treatment modalities. imatinib is now frontline therapy for CML but resistance is increasingly encountered. According to one study, the estimated 2-year incidence of resistance to imatinib mesylate was 80% in blastic phase, 40% to 50% in accelerated phase, and 10% in chronic phase post-interferon-α failure (Kantarjian et al, Blood, 101 (2):473-475 (2003). N-(2-chloro-6-methylphenyl)-2-[[6-[4-(2-hydroxyethyl) 1-piperazinyl]-2-methyl-4-pyrimidinyl]amino]-5-thiazolecarboxamide is an ATP-competitive, dual SRC/ABL inhibitor (Lombardo, L. J., et al., J. Med. Chem., 47:6658-6661 (2004)). Notably, N-(2-chloro-6-methylphenyl)-2-[[6-[4-(2-hydroxyethyl)-1-piperazinyl]-2-methyl-4-pyrimidinyl]amino]-5-thiazolecarboxamide has been shown to inhibit BCR-ABL imatinib-resistant mutations that are found in some CML patients with acquired clinical resistance to imatinib. On account of the demonstration that patients harboring different Src mutations, particularly BCR/ABL mutations, have varying degrees of resistance and/or sensitivity to imatinib and N-(2-chloro-6-methylphenyl)-2-[[6-[4-(2-hydroxyethyl)-1-piperazinyl]-2-methyl-4-pyrimidinyl]amino]-5-thiazolecarboxamide, respectively, the inventors of the present invention describe for the first time methods to identify patients who may most benefit from either increased doses of N-(2-chloro-6-methylphenyl)-2-[[6-[4-(2-hydroxyethyl)-1-piperazinyl]-2-methyl-4-pyrimidinyl]amino]-5-thiazolecarboxamide, increased dosing frequency of N-(2-chloro-6-methylphenyl)-2-[[6-[4-(2-hydroxyethyl)-1-piperazinyl]-2-methyl-4-pyrimidinyl]amino]-5-thiazolecarboxamide, or the combination of N-(2-chloro-6-methylphenyl)-2-[[6-[4-(2-hydroxyethyl)-1-piperazinyl]-2-methyl-4-pyrimidinyl]amino]-5-thiazolecarboxamide, with other protein tyrosine kinase inhibitors, or other agents, by assessing the patients expression level of MDR-1 wherein overexpression may warrant the administration of a higher dose or dosing frequency of N-(2-chloro-6-methylphenyl)-2-[[6-[4-(2-hydroxyethyl)-1-piperazinyl]-2-methyl-4-pyrimidinyl]amino]-5-thiazolecarboxamide, or combination.

The structure and use of N-(2-chloro-6-methylphenyl)-2-[[6-[4-(2-hydroxyethyl)-1-piperazinyl]-2-methyl-4-pyrimidinyl]amino]-5-thiazolecarboxamide as an anticancer agent is described in Lombardo, L. J., et al., J. Med. Chem., 47:6658-6661 (2004) and is described in the following US patents and pending applications, incorporated herein by reference: U.S. Pat. No. 6,596,746, granted Jul. 22, 2003; U.S. Ser. No. 10/395,503, filed Mar. 24, 2003.

The structure and use of imatinib as an anticancer agent is described in B. J. Druker et al., N. Engl. J. Med. 344, 1031 (2001) and S. G. O'Brien et al., N. Engl. J. Med. 348, 994 (2003).

The terms “combination”, and “combination of N-(2-chloro-6-methylphenyl)-2-[[6-[4-(2-hydroxyethyl)-1-piperazinyl]-2-methyl-4-pyrimidinyl]amino]-5-thiazolecarboxamide” as used herein refers to a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide with AMN-107, PD180970, CGP76030, AP23464, SKI 606, NS-187, AZD0530, and/or ARIAD; any combinations specifically disclosed in co-owned U.S. Ser. No. 60/670,744, filed Apr. 13, 2005 (hereby incorporated herein by reference); and/or any compounds disclosed and referenced in Deininger et al (Blood, 105(7):2640-2653 (2005); hereby incorporated by reference in its entirety) which include, but are not limited to IFN, pegylated IFN, homoharringtonine, cytabine, hydroxyurea, farnesyl transferase inhibitors, lonafarnib, tipifamib, MEK1 inhibitors, PD98059, RAF-1 inhibitors, BAY43-9006, PI3 kinase inhibitors, LY294002, mTOR inhibitors, rapamycin, cyclin-dependent kinase inhibitors, favopiridol; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and ATP non-competitive inhibitors ONO12380; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and Aurora kinase inhibitor VX-680; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and p38 MAP kinase inhibitor BIRB-796; a combination of N-(2-chloro-6-methylphenyl)-2-[[6-[4-(2-hydroxyethyl)-1-piperazinyl]-2-methyl-4-pyrimidinyl]amino]-5-thiazolecarboxamide with any number of BCR-ABL inhibitors; a combination of any compounds disclosed and/or referenced in La Rosee et al (Leukemia, 16:1213-1219 (2002), hereby incorporated by reference in its entirety); or pharmaceutically acceptable salts thereof, and optionally in therapeutically effective amounts thereof.

For use herein, a “BCR-ABL inhibitor” refers to any molecule or compound that can partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bioorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trail a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity against CML cell (Golas et al., Cancer Research (2003) 63, 375-381). AZD0530 is a dual Abl/Src kinase inhibitor that is in ongoing clinical trials for the treatment of solid tumors and leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O'Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

The terms “treating”, “treatment” and “therapy” as used herein refer to curative therapy, prophylactic therapy, preventative therapy, and mitigating disease therapy.

The phrase “more aggressive dosing regimen”, “increased dosing frequency regimen”, as used herein refers to a dosing regimen that necessarily exceeds the basal and/or prescribed dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide either due to an increased frequency of administration, increased or escalated dose, or the route of administration which may result in an increased bio-available level of N-(2-chloro-6-methylphenyl)-2-[[6-[4-(2-hydroxyethyl)-1-piperazinyl]-2-methyl-4-pyrimidinyl]amino]-5-thiazolecarboxamide. Non-limiting examples of such dosing regimens may be found by reference to U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; and/or dosing regimens outlined in Deininger et al (Blood, 105(7):2640-2653 (2005); hereby incorporated by reference in its entirety); which are hereby incorporated herein by reference.

The phrase “MDR-1 overexpression” is meant to encompass a level of expression of MDR-1 mRNA, transcripts, and/or protein that is at least about 1, 2, 3, 4, 5, 6, 7, 8, 9, 10 fold or more higher than a reference or normal level of MDR-1 expression. However, modest levels of increased expression, such as about 0.1, 0.2, 0.3, 0.4, 0.5, 0.6, 0.7, 0.8, 0.9 fold higher levels than a reference or normal level of MDR-1 expression are also encompassed by this phrase.

Treatment regimens can be established based upon the detection of MDR-1 overexpression. For example, the invention encompasses screening cells from an individual who may suffer from, or is suffering from, a disorder that is commonly treated with a kinase inhibitor. Such a disorder can include myeloid leukemia or disorders associated therewith, or cancers described herein. The cells of an individual are screened, using methods known in the art, for identification of MDR-1 overexpression.

If MDR-1 overexpression is found in the cells from said individual, treatment regimens can be developed appropriately. For example, MDR-1 overexpression can indicate that said cells are or will become at least partially resistant to commonly used kinase inhibitors, including BCR-ABL inhibitors. For example, MDR-1 overexpression can indicate that the cells in an individual are or are expected to become at least partially resistant to treatment with a kinase inhibitor such as N-(2-chloro-6-methylphenyl)-2-[[6-[4-(2-hydroxyethyl) 1-piperazinyl]-2-methyl-4-pyrimidinyl]amino]-5-thiazolecarboxamide and that administration of higher doses of the same may be warranted. As disclosed herein, in such cases, treatment can include the use of an increased dosing frequency or increased dosage of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and another kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; any other combination disclosed herein; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. In one aspect, an increased level of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual.

A therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof can be orally administered as an acid salt of N-(2-chloro-6-methylphenyl)-2-[[6-[4-(2-hydroxyethyl)-1-piperazinyl]-2-methyl-4-pyrimidinyl]amino]-5-thiazolecarboxamide. The actual dosage employed can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof (and Compound I salt) can be determined by one of ordinary skill in the art, and includes exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, per day, which can be administered in a single dose or in the form of individual divided doses, such as from 1, 2, 3, or 4 times per day. In certain embodiments, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

A treatment regimen is a course of therapy administered to an individual suffering from a protein kinase associated disorder that can include treatment with one or more kinase inhibitors, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. As disclosed herein, for example, cells from an individual suffering from a protein kinase associated disorder can be found to develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide. Based upon the present discovery that such cells can be sensitive to combination therapy or a more aggressive dosage or dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, a treatment regimen can be established that includes treatment with the combination either as a monotherapy, or in combination with another kinase inhibitor, or in combination with another agent as disclosed herein. Additionally, the combination can be administered with radiation or other known treatments.

Therefore the present invention includes a method for establishing a treatment regimen for an individual suffering from a protein tyrosine kinase associated disorder or treating an individual suffering from a protein tyrosine kinase disorder comprising determining whether a biological sample obtained from an individual has MDR-1 overexpression, and administering to the subject an appropriate treatment regimen based on whether MDR-1 overexpression is present. The determination can be made by any method known in the art, for example, by screening said sample of cells for the presence of MDR-1 overexpression or by obtaining information from a secondary source that the individual has MDR-1 overexpression.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Pharmaceutical compositions for use in the present invention can include compositions comprising one or a combination of BCR-ABL inhibitors in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving N-(2-chloro-6-methylphenyl)-2-[[6-[4-(2-hydroxyethyl)-1-piperazinyl]-2-methyl-4-pyrimidinyl]amino]-5-thiazolecarboxamide useful in practicing the present invention are described in U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; which are hereby incorporated herein by reference in their entirety and for all purposes.

A “therapeutically effective amount” of an inhibitor of BCR-ABL can be a function of whether MDR-1 overexpression is present. A therapeutically relevant dose of N-(2-chloro-6-methylphenyl)-2-[[6-[4-(2-hydroxyethyl)-1-piperazinyl]-2-methyl-4-pyrimidinyl]amino]-5-thiazolecarboxamide for patients having MDR-1 overexpression, for example, could range anywhere from 1 to 14 fold higher than the typical dose. Accordingly, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide for any of the BCR-ABL-associated or protein tyrosine kinase associated disorder in which MDR-1 overexpression is present can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide can be, for example, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose.

Wherever the term “N-(2-chloro-6-methylphenyl)-2-[[6-[4-(2-hydroxyethyl)-1-piperazinyl]-2-methyl-4-pyrimidinyl]amino]-5-thiazolecarboxamide” (e.g., Compound I) is used herein, it is understood (unless otherwise indicated) that the compound N-(2-chloro-6-methylphenyl)-2-[[6-[4-(2-hydroxyethyl)-1-piperazinyl]-2-methyl-4-pyrimidinyl]amino]-5-thiazolecarboxamide having the following structure (I):

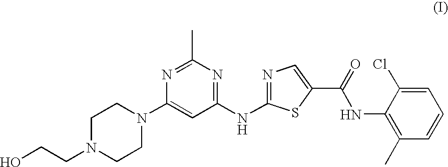

is intended, as well as all pharmaceutically acceptable salts thereof. Compound (I) is also referred to as N-(2-chloro-6-methylphenyl)-2-((6-(4-(2-hydroxyethyl)-1-piperazinyl)-2-methyl-4-pyrimidinyl)amino)-1,3-thiazole-5-carboxamide in; accordance with IUPAC nomenclature. Use of the term encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference). Pharmaceutical compositions of N-(2-chloro-6-methylphenyl)-2-[[6-[4-(2-hydroxyethyl)-1-piperazinyl]-2-methyl-4-pyrimidinyl]amino]-5-thiazolecarboxamide include all pharmaceutically acceptable compositions comprising N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference.

Wherein the term “a farnysyl transferase inhibitor” (e.g., Compound II) herein, it is understood (unless otherwise indicated) that the compound have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt, is an anti-cancer agent. The compound of formula (II) is a cytotoxic FT inhibitor which is known to kill non-proliferating cancer cells preferentially. The compound of formula (II) may further be useful in killing stem cells.

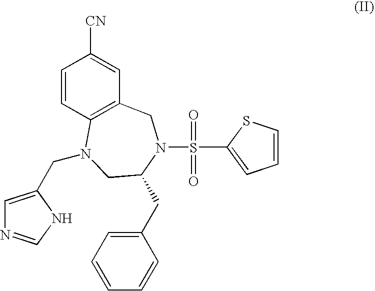

The compound of formula (II), its preparation, and uses thereof are described in U.S. Pat. No. 6,011,029, which is herein incorporated by reference. The uses of the compound of formula (II) are also described in WO2004/015130, published Feb. 19, 2004, which is herein incorporated by reference.

“Protein tyrosine kinase-associated disorders” are those disorders which result from aberrant tyrosine kinase activity, and/or which are alleviated by the inhibition of one or more of these enzymes. Disorders included in the scope of the present invention may include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various cancers are also included within the scope of protein tyrosine kinase-associated disorders including (but not limited to) the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma.

“Protein tyrosine kinase-associated disorders” may also include those disorders which result from BCR-ABL activity, including mutant BCR-ABL activity, and/or which are alleviated by the inhibition of BCR-ABL, including mutant BCR-ABL, expression and/or activity. A reciprocal translocation between chromosomes 9 and 22 produces the oncogenic BCR-ABL fusion protein. The phrase “Protein tyrosine kinase-associated disorders” is inclusive of “mutant BCR-ABL associated disorders” and “BCR-ABL associated disorders”.

The present invention provides methods of determining responsiveness of an individual having a protein tyrosine kinase-associated disorder to a certain treatment regimen and methods of treating an individual having a protein tyrosine kinase-associated disorders.

Disorders included in the scope of the present invention include, for example, leukemias, including, for example, chronic myeloid leukemia, acute lymphoblastic leukemia, and Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ALL, AML, imatinib-resistant CML, imatinib-intolerant CML, accelerated CML, lymphoid blast phase CML.

A “solid tumor” includes, for example, sarcoma, melanoma, carcinoma, or other solid tumor cancer.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ALL).

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### Detection Method

The invention provides methods of screening a biological sample from an individual for the presence of overexpressed MDR-1 expression relative to a standard level of MDR-1 expression, as well as methods for treating individuals who are identified as having an MDR-1 overexpression.

Methods of measuring the expression level of MDR-1 in cells are known in the art, including, but not limited to PCR, RT-PCR, ELISA, Western Blots, hybridization, mass spectrometry, etc. Standard molecular biology techniques are contemplated for precisely determining the expression level of MDR-1 in the cells of a given individual.

Antibodies directed at MDR-1 can be useful in the diagnosis, and/or prognosis of other cancers in order to facilitate the measuring the level of MDR-1 expression in a given sample. Accordingly the invention provides various immunological assays useful for the detection and quantification of MDR-1 proteins and polypeptides. Such assays generally comprise one or more MDR-1 directed antibodies capable of recognizing and binding a MDR-1 protein, as appropriate, and can be performed within various immunological assay formats well known in the art, including, for example, various types of radioimmunoassays, enzyme-linked immunosorbent assays (ELISA), enzyme-linked immunofluorescent assays (ELIFA), and the like. In addition, immunological imaging methods capable of detecting cancer cells are also provided by the invention including, for example, imaging methods using labeled MDR-1 antibodies. Such assays can be used clinically in the detection, monitoring, and prognosis of cancers.

Accordingly, the present invention provides methods of assaying for the presence of a MDR-1polypeptide of the present invention. By way of example only, in certain embodiments, an antibody raised against the fragment, or other binding moiety capable of specifically binding to the target analyte, is immobilised onto a solid substrate to form a first complex and a biological test sample from a patient is brought into contact with the bound molecule. After a suitable period of incubation, for a period of time sufficient to allow formation of an antibody-secondary complex, a second antibody labeled with a reporter molecule capable of producing a detectable signal is then added and incubated, allowing sufficient time for the formation of a tertiary complex. Any unreacted material is washed away, and the presence of the tertiary complex is determined by observation of a signal produced by the reporter molecule. The results may either be qualitative, by simple observation of the visible signal or may be quantitated by comparison with a control sample containing known amounts of hapten. Variations of this assay include a simultaneous assay, in which both sample and labelled antibody are added simultaneously to the bound antibody, or a reverse assay in which the labelled antibody and sample to be tested are first combined, incubated and then added simultaneously to the bound antibody. These techniques are well known to those skilled in the art, and the possibility of variations will be readily apparent.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecule in this type of assay are either enzymes, fluorophores or radionuclide containing molecules (i.e. radioisotopes).

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing the molecule to the insoluble carrier.

The expression profiles of MDR-1 can be used as diagnostic markers for disease states. The status of MDR-1 gene products in patient samples can be analyzed by a variety protocols that are well known in the art including the following non-limiting types of assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, microfluldic genotype assays, immunohistochemical analysis, the variety of Northern blotting techniques including in situ hybridization, RT-PCR analysis (for example on laser capture micro-dissected samples), western blot analysis, tissue array analysis, and any other methods known in the art or described elsewhere herein. Probes and primers can be designed so as to be specific to the MDR-1 sequence, segments and complementary sequences thereof.

Additionally, the invention provides assays for the detection of MDR-1 polynucleotides in a biological sample, such as cell preparations, and the like. A number of methods for amplifying and/or detecting the presence of MDR-1 polynucleotides are well known in the art and can be employed in the practice of this aspect of the invention.

In certain embodiments, a method for detecting MDR-1 in a biological sample comprises producing cDNA from the sample by reverse transcription using at least one primer; amplifying the cDNA so produced using MDR-1 polynucleotides as sense and antisense primers to amplify MDR-1 cDNAs therein; and detecting the presence and expression level of the amplified MDR-1 cDNA. Any number of appropriate sense and antisense probe combinations can be designed from the nucleotide sequences provided for a MDR-1 and used for this purpose.

The invention also provides assays for detecting the presence of a mutant BCR-ABL kinase protein in a biological sample. Methods for detecting a mutant BCR-ABL kinase protein are also well known and include, for example, immunoprecipitation, immunohistochemical analysis, Western Blot analysis, molecular binding assays, ELISA, ELIFA and the like. For example, in one embodiment, a method of detecting the presence of a mutant BCR-ABL kinase protein in a biological sample comprises first contacting the sample with a BCR-ABL antibody, a mutant BCR-ABL kinase-reactive fragment thereof, or a recombinant protein containing an antigen binding region of a mutant BCR-ABL kinase antibody; and then detecting the binding of mutant BCR-ABL kinase protein in the sample thereto.

Methods for identifying a cell that overexpresses MDR-1 are also provided. In one embodiment, an assay for identifying a cell that overexpresses MDR-1 comprises detecting the presence of MDR-1 mRNA in the cell and comparing the level of expression to a standard cell that expresses MDR-1 at a standard level. Methods for the detection of particular mRNAs in cells are well known and include, for example, hybridization assays using complementary DNA probes (such as in situ hybridization using labeled MDR-1 kinase riboprobes, Northern blot and related techniques) and various nucleic acid amplification assays (such as RT-PCR using complementary primers specific for MDR-1, and other amplification type detection methods, such as, for example, branched DNA, SISBA, TMA and the like).

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be an antibody or polynucleotide specific for measuring MDR-1 expression. Where the kit utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting a BCR-ABL kinase and/or mutant BCR-ABL kinases. Specifically contemplated by the invention is a kit comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and another protein tyrosine kinase inhibitor, such as, imatinib, AMN107, PD180970, GGP76030, AP23464, SKI 606, NS-187, and/or AZD0530; an increased dose and/or dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, relative a treatment regimen suitable for such other forms of such BCR-ABL kinase (e.g., wild-type); and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof disclosed herein, useful in treating mammals suffering from a BCR-ABL associated disorder, including mutant BCR-ABL associated disorder. For example, kits useful in identifying a mutant BCR-ABL kinase in a mammalian patient (e.g., a human) suffering from a cancer that is completely or partially resistant to, or has developed complete or partial resistance to, N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, imatinib, or another protein tyrosine kinase inhibitor and where said kits also comprise a therapeutically effective amount of the combination or increased dose or dosing regimen, are contemplated herein.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The present invention is not to be limited in scope by the embodiments disclosed herein, which are intended as single illustrations of individual aspects of the invention; and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1. Daley G Q, Van Etten R A, Baltimore D. Induction of chronic myelogenous leukaemia in mice by the P210 bcr/abl gene of the Philadelphia chromosome. Science. 1990; 247:824-830.
2. Heisterkamp N, Jenster G, ten Hoeve J, Zovich D, Pattengale P K, Groffen J. Acute leukaemia in bcr/abl transgenic mice. Nature. 1990; 344:251-253.
3. Faderl S, Kantarjian H M, Thomas D A, et al. Outcome of Philadelphia chromosome-positive adult acute lymphoblastic leukemia Leuk Lymphoma 2000; 36:263-273.
4. Lugo T G, Pendergast A M, Muller A J, Witte O N. Tyrosine kinase activity and transformation potency of bcr-abl oncogene products. Science. 1990; 247:1079-1082.
5. Druker B J, Talpaz M, Resta D J, et al. Efficacy and Safety of a Specific Inhibitor of the BCR-ABL Tyrosine Kinase in Chronic Myeloid Leukemia N Engl J Med. 2001; 344:1031-1037.
6. Kantarjian H M, Sawyer C, Hochhaus A, et al. Hematologic and cytogenetic responses to imatinib mesylate in chronic myelogenous leukemia N Engl J Med. 2002; 346:645-652.
7. Druker B J, Sawyers C L, Kantarjian H, et al. Activity of a Specific Inhibitor of the BCR-ABL Tyrosine Kinase in the Blast Crisis of Chronic Myeloid Leukemia and Acute Lymphoblastic Leukemia with the Philadelphia Chromosome. N Engl J Med. 2001; 344:1038-1042.
8. Talpaz M, Silver R T, Druker B J, et al. Imatinib induces durable hematologic and cytogenetic responses in patients with accelerated phase chronic myeloid leukemia: results of a phase 2 study. Blood. 2002; 99:1928-1937.
9. Sawyers C, Hochhaus A, Feldman E, et al. Imatinib induces hematologic and cytogenetic responses in patients with chronic myelogenous leukemia in myeloid blast crisis: results of a phase II study. Blood 2002; 99:3530-3539.
10. Ottmann O G, Druker B J, Sawyers C L, et al. A phase 2 study of imatinib in patients with relapsed or refractory Philadelphia chromosome-positive acute lymphoid leukemias. Blood 2002; 100:1965-1971.
11. Kantarjian H, Schiffer C, Sawyers C, et al. Imatinib (Gleevec®) maintains favorable long-term outcomes in chronic-phase chronic myeloid leukemia (CML) for patients failing interferon-alpha (IFN)—follow-up of a Phase II study. Blood 2003; 102:905a (Abstract 3368).
12. Talpaz M, Goldman J, Sawyers C, et al. High dose imatinib (STI571, Gleevec®) provides durable long-term outcomes for patients (pts) with chronic myeloid leukemia (CML) in accelerated phase (AP) or myeloid blast crisis (BC): follow-up of the Phase II studies. Blood. 2003; 102:905a-906a (Abstract 3369).
13. Gone M E, Mohammed M, Ellwood K, et al. Clinical Resistance to STI-571 Cancer Therapy Caused by BCR-ABL Gene Mutation or Amplification. Science. 2001; 293:876-880.
14. Donato N J, Wu J Y, Stapley J, et al. BCR-ABL independence and LYN kinase overexpression in chronic myelogenous leukemia cells selected for resistance to STI571. Blood. 2003; 101:690-698.
15. Gambacorti-Passerini C, Barni R, le Coutre P, et al. Role of {alpha}1 Acid Glycoprotein in the In Vivo Resistance of Human BCR-ABL+Leukemic Cells to the Abl Inhibitor STI571. J. Natl Cancer Inst. 2000; 92:1641-1650.
16. Thomas J, Wang L, Clark R E, Pirmohamed M. Active transport of imatinib into and out of cells: implications for drug resistance. Blood. 2004; 104:3739-3745.
17. Illmer T, Schaich M, Platzbecker U, et al. P-glycoprotein-mediated drug efflux is a resistance mechanism of chronic myelogenous leukemia cells to treatment with imatinib mesylate. Leukemia 2004; 18:401-408.
18. Warmuth M, Simon N, Mitina O, et al. Dual-specific Src and Abl kinase inhibitors, PP1 and CGP76030, inhibit growth and survival of cells expressing imatinib mesylate-resistant Bcr-Abl kinases. Blood. 2003; 101:664-672.
19. Warmuth M, Damoiseaux R, Liu Y, Fabbro D, Gray N. SRC family kinases: potential targets for the treatment of human cancer and leukemia Curr Pharm Des. 2003; 9:2043-2059.
20. Tipping A J, Baluch S, Barnes D J, et al. Efficacy of dual-specific Bcr-Abl and Src-family kinase inhibitors in cells sensitive and resistant to imatinib mesylate. Leukemia 2004; 18:1352-1356.
21. Hu Y, Liu Y, Pelletier S, et al. Requirement of Src kinases Lyn, Hck and Fgr for BCR-ABL1-induced B-lymphoblastic leukemia but not chronic myeloid leukemia. Nature Genetics. 2004; 36:453-461.
22. Lombardo L J, Lee F Y, Chen P, et al. Discovery of N-(2-Chloro-6-methylphenyl)-2-(6-(4-(2-hydroxyethyl)piperazin-1-yl)-2-methylpyrmidin-4-ylamino)thiazole-5-carboxamide (BMS-54825), a Dual Src/Abl Kinase Inhibitor with Potent Anti-Tumor Activity in Preclinical Assays. J Med. Chem. 2004; 47:6658-6661.
23. Shah N P, Tran C, Lee F Y, Chen P, Norris D, Sawyers C L. Overriding Imatinib Resistance with a Novel ABL Kinase Inhibitor. Science. 2004; 305:399-401.
24. O'Hare T, Walters D K, Stoffregen E P, et al. In vitro Activity of Bcr-Abl Inhibitors AMN107 and BMS-354825 against Clinically Relevant Imatinib-Resistant Abl Kinase Domain Mutants. Cancer Res. 2005; 65:4500-4505.
25. Sawyers C L, Kantarjian H, Shah N, et al. Dasatinib (BMS-354825) in patients with chronic myeloid leukemia (CML) and Philadelphia-chromosome positive acute lymphoblastic leukemia (Ph+ALL) who are resistant or intolerant to imatinib: update of a Phase I study [abstract]. Blood. 2005; 106:16a (Abstract 38).
26. Breitenstein W, Furet P, Jacob S, Manley P W. Inhibitors of tyrosine kinases; 2003.
27. Peng B, Hayes M, Resta D, et al. Pharmacokinetics and Pharmacodynamics of Imatinib in a Phase I Trial With Chronic Myeloid Leukemia Patients. J Clin Oncol. 2004; 22:935-942.
28. Tsuruo T, Iida-Saito H, Kawabata H, Oh-hara T, Hamada H, Utakoji T. Characteristics of resistance to adriamycin in human myelogenous leukemia K562 resistant to adriamycin and in isolated clones. Jpn J Cancer Res. 1986; 77:682-692.
29. Cole P A, Grace M R, Philips R S, Burn P, Walsh C T. The role of the catalytic base in the protein tyrosine kinase Csk. Journal of Biological Chemistry. 1995; 270:22105-22108.
30. Bieth J G. Theoretical and practical aspects of proteinase inhibition kinetics. Methods Enzymol. 1995; 248:59-84.
31. Lee F Y F, Borzilleri R, Fairchild C R, et al. BMS-247550: A novel epothilone analog with a mode of action similar to paclitaxel but possessing superior antitumor activity. Clin Cancer Res. 2001; 7:1429-1437.
32. Gehan E A. A generalized Wilcoxon test for comparing arbitrarily singly-censored samples. Biometrika 1965; 52:203-223.
33. Shah N P, Nicoll J M, Nagar B, et al. Multiple BCR-ABL kinase domain mutations confer polyclonal resistance to the tyrosine kinase inhibitor imatinib (STI571) in chronic phase and blast crisis chronic myeloid leukemia. Cancer Cell. 2002; 2:117-125.
34. Weisberg E, Manley P W, Breitenstein W, et al. Characterization of AMN107, a selective inhibitor of native and mutant Ber-Abl. Cancer Cell. 2005; 7:129-141.
35. Sugimoto Y, Roninson I B, Tsuruo T. Decreased expression of the amplified mdr1 gene in revertants of multidrug-resistant human myelogenous leukemia K562 occurs without loss of amplified DNA. Mol Cell Biol. 1987; 7:4549-4552.
36. le Coutre P, Mologni L, Cleris L, Buchdunger E, Giardini R. In vivo eradication of human BCR/ABL-positive leukemia cells with an ABL kinase inhibitor. J Natl Cancer Inst. 1999; 9:163-168.
37. Schindler T, Bornmann W, Pellicena P, Miller W T, Clarkson B, Kuriyan J. Structural Mechanism for STI-571 Inhibition of Abelson Tyrosine Kinase. Science. 2000; 289:1938-1942.
38. Tokarski J S, Newitt J, Lee F Y, et al. The Crystal Structure of Abl Kinase with BMS-354825, a Dual SRC/ABL Kinase Inhibitor. Blood 2004; 104:106a (Abstract 553).
39. Azam M, Latek R R, Daley G Q. Mechanisms of Autoinhibition and STI-571/Imatinib Resistance Revealed by Mutagenesis of BCR-ABL. Cell. 2003; 112:831-843.
40. Mahon F-X Belloc F, Lagarde V, et al. MDR1 gene overexpression confers resistance to imatinib mesylate in leukemia cell line models. Blood 2003; 101:2368-2373.
41. Mukai M, Che X-F, Furukawa T, et al. Reversal of the resistance to STI571 in human chronic myelogenous leukemia K562 cells. Cancer Sci. 2003; 94:557-563.
42. Dai H, Marbach P, Lemaire M, Hayes M, Elmquist W. Distribution of STI-571 to the brain is limited by p-glycoprotein-mediated efflux. J Pharmacol Exp Ther. 2003; 304:1085-1092.
43. Bujassoum S, Rifkind J, Lipton J. Isolated central nervous system relapse in lymphoid blast crisis chronic myeloid leukemia and acute lymphoblastic leukemia in patients on imatinib therapy. Leuk Lymphoma 2004; 45:401-403.
44. Leis J, Stepan D, Curtin P, et al. Central nervous system failure in patients with chronic myelogenous leukemia lymphoid blast crisis and Philadelphia chromosome positive acute lymphoblastic leukemia treated with imatinib (STI-571). Leuk Lymphoma. 2004; 45:695-698.
45. Matsuda M, Morita Y, Shimada T, et al. Extramedullary blast crisis derived from 2 different clones in the central nervous system and neck during complete cytogenetic remission of chronic myelogenous leukemia treated with imatinib mesylate. Int J Hematol. 2005; 81:307-309.
46. Wild R, Castaneda S, Flefleh C, et al. BMS-354825, a Dual SRC/ABL Kinase Inhibitor, Displays Potent Anti-Tumor Activity in a Model of Intracranial CML Growth. Blood. 2004; 104:549a (abstract 1988).
47. van der Pol M A, Broxterman H J, Pater J M, et al. Function of the ABC transporters, P-glycoprotein, multidrug resistance protein and breast cancer resistance protein, in minimal residual disease in acute myeloid leukemia Haematologica 2003; 88:134-147.
48. Copland M, Hamilton A, Baird J W, et al. Dasatinib (BMS-354825) has increased activity against BCR-ABL compared to imatinib in primary CML cells in vitro, but does not eradicate quiescent CML stem cells [abstract]. Blood. 2005; 106:205a (Abstract 695).
49. Burgess M R, Skaggs B J, Shah N P, Lee F Y, Sawyers C L. Comparative analysis of two clinically active BCR-ABL kinase inhibitors reveals the role of conformation-specific binding in resistance. Proc Natl Acad. Sci. 2005; 102:3395-3400.
50. Mambou P, Romanski A, Bug G, et al. The SRC-kinase inhibitor AZD0530 efficiently counteracts the transformation potential of BCR/ABL by targeting its kinase activity. Eur J Cancer Suppl. 2004; 2:109 (Abstract 364).
51. Hochhaus A, Baccarani M, Sawyers C, et al. Efficacy of dasatinib in patients with chronic phase Philadelphia chromosome-positive CML resistant or intolerant to imatinib: first results of the CA180013 ‘START-C’ Phase II study [abstract]. Blood 2005; 106:17a (Abstract 41).
52. Guilhot F, Apperley J F, Shah N, et al. A Phase II study of dasatinib in patients with accelerated phase chronic myeloid leukemia (CML) who are resistant or intolerant to imatinib: first results of the CA180005 ‘START-A’ study [abstract]. Blood. 2005; 106:16a (Abstract 39).
53. Talpaz M, Rousselot P, Kim D W, et al. A Phase II study of dasatinib in patients with chronic myeloid leukemia (CML) in myeloid blast crisis who are resistant or intolerant to imatinib: first results of the CA180006 ‘START-B’ study [abstract]. Blood. 2005; 106:16a (Abstract 40).
54. Ottmann O G, Martinelli G, Dombret H, et al. A Phase II study of dasatinib in patients with chronic myeloid leukemia (CML) in lymphoid blast crisis or Philadelphia-chromosome positive acute lymphoblastic leukemia (Ph+ALL) who are resistant or intolerant to imatinib: the ‘START-’CA180015 study [abstract]. Blood. 2005; 106:17a (Abstract 42).
55. Bornhauser, M., Jenke, A., et al. (2004) CNS blast crisis of chronic myelogenous leukemia in a patient with a major cytogenetic response in bone marrow associated with low levels of imatinib mesylate and its N-desmethylated metabolite in cerebral spinal fluid. Annals of Hematology 83: 401-402.

### EXAMPLES

### Example 1

### Method of Determining Sensitivity of Imatinib Sensitive and Resistant Cells to Dasatinib In Vitro

Previous studies have shown that dasatinib demonstrates potent, nanomolar inhibition of BCR-ABL kinase activity²². In addition, previous studies have also demonstrated dasatinib's potent inhibition of 18 of 19 common mutations in BCR-ABL that are known to confer imatinib resistance preclinically, both in vitro and in vivo,^{23,24} as well as clinically in a Phase I dose finding study. Comparisons between imatinib and dasatinib were performed in both the imatinib sensitive cell line K562 (erythromyeloblastoid CML) and imatinib resistant cell line K562R (erythromyeloblastoid CML). Experiments were performed as follows:

### Compounds, Cell Lines and Xenograft Models

Dasatinib was generated by Bristol-Myers Squibb laboratories (Princeton, N.J.). Imatinib was purchased from Novartis (Basel, Switzerland). Two cell lines were used: K562 (erythromyeloblastoid CML) and K562/ADM (K562 cells over-expressing MDR-1). Imatinib-resistant cells (K562/STI-571/R) were derived from the K562 cell line by growth in the presence of imatinib, as described elsewhere.²⁷ K562/ADM cells were obtained from Japan Health Sciences Foundation (Osaka, Japan)²⁸. For in vivo experiments, human tumor xenografts (propagated from CML cell lines) were maintained in Balb/c nu/nu nude or SCID mice (Harlan, Ind.) and propagated as subcutaneous transplants using tumor fragments from donor mice.

### In Vitro Proliferation/Cytotoxicity Assays

In vitro cytotoxicity was assessed using a tetrazolium-based colorimetric assay, as previously described.³¹ Cells were incubated for 72 hours at 37° C. with kinase inhibitor, then plated at a density of 1-4×10⁴ cells/well in 6-well culture plates and grown overnight. Cell growth was determined by direct counting using a Coulter Channelyzer (Beckman Coulter, Miami, Fla.). To determine cell death, propidium iodide was added to a final concentration of 5 μg/mL. Data was acquired by BD FACS calibur (Becton Dickinson, San Jose, Calif.) using standard filters and analyzed using Flow Jo software (Tree Star, Inc, San Carlos, Calif.).

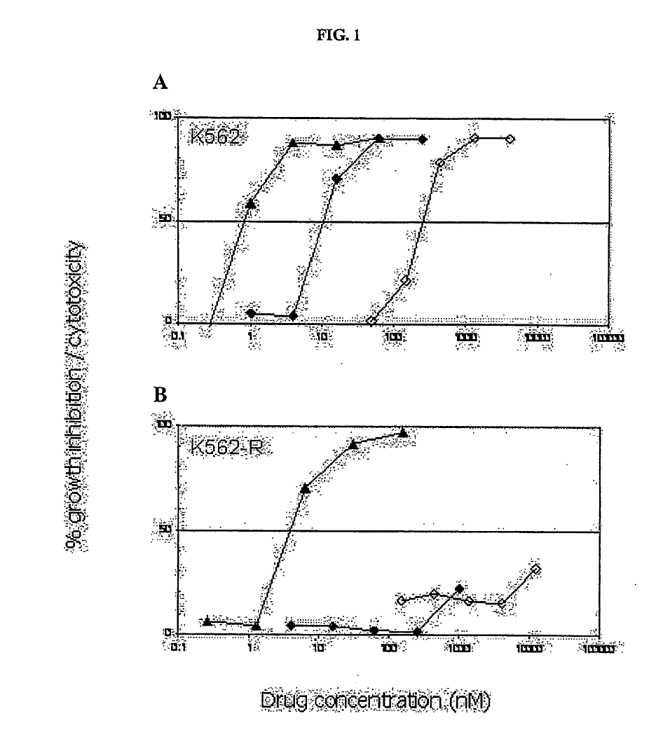

Results of the comparison experiments between imatinib and dasatinib in both the imatinib sensitive cell line K562 (erythromyeloblastoid CML) and imatinib resistant cell line K562R (erythromyeloblastoid CML) are provided in FIGS. 1A-B. As shown, K562-R demonstrated extreme resistance to imatinib with an IC₅₀ of >12.15 □M (versus 217 nM in K562 cells), representing a resistance factor of >56 fold. In contrast, K562-R was modestly cross-resistant to dasatinib, the IC₅₀ being only 6-fold higher in the resistant line versus K562 cells (4.4 nM versus 0.7 nM). Donato et al. established imatinib resistance is due to activation of LYN and the concomitant loss of dependence on BCR-ABL for growth and survival in these cells.¹⁴ However, since dasatinib also inhibits LYN, the 6 fold difference in sensitivity between K562 and K562R was unclear. Overexpression of the p-glycoprotein effux pump, MDR-1, was considered as a possible mechanism.

### Example 2

### Method of Determining the Relation Between MDR-1 Overexpression to the Modest Differential Sensitivity of Imatinib Sensitive and Resistant Cells to Dasatinib In Vitro

It has been reported that imatinib resistance in CML cells, particularly primitive leukemic cells with stem-cell like properties, may be associated with increased expression of MDR-1, the gene encoding the 170 Kd p-glycoprotein (Pgp) efflux pump.^{16,17}

To investigate whether dasatinib can be effective in the treatment of leukemic cells with Pgp overexpression, the cytotoxicity of dasatinib against K562/ADM, a cell line that was selected for resistance to adriamycin and whose resistance is known to be in large part due to increased expression of Pgp, was evaluated.³⁵

Experiments were performed as described in Example 1 except that K562/ADM cells were used. K562/ADM was 60-fold more resistant to adriamycin versus K562 cells (IC₅₀ 1252 nM versus 21 nM in K562 cells). In contrast, dasatinib, with an IC₅₀ of 3 nM, was only 6-fold less active against K562/ADM than the parent K562 line (IC₅₀ 0.5 nM), indicating that dasatinib can substantially, although not completely, overcome the MDR resistance inherent in the K562/ADM cells. The results also suggest measuring MDR-1 expression in patients may serve as a useful tool in identifying patients who may be in need of modestly higher doses of dasatinib to offset the slight decrease in dasatinib sensitivity in cells overexpressing MDR-1.

### Example 3

### Method of Determining Sensitivity of Imatinib Sensitive and Resistant Cells to Dasatinib In Vivo

In order to assess whether the in vitro anti-proliferative activity of dasatinib against imatinib-resistant CML cell lines also translated into in vivo efficacy, experiments were performed using mouse xenograft models of imatinib-resistant CML. Briefly, the experiments were performed as follows:

### Tumor Response in Mouse Xenograft Models

Tumors implanted in mouse xenograft models were allowed to grow to a pre-determined size of 100-300 mg (tumors outside the range were excluded) and animals were evenly distributed to treatment and control groups. Treated animals were checked daily for treatment-related toxicity/mortality. Each group of animals (typically eight mice per treatment or control group) was weighed before the treatment initiation (Wt1) and again following the last treatment dose (Wt2). The difference in body weight (Wt2−Wt1) provides a measure of treatment-related toxicity. Tumor weights (mg) were estimated from the formula: tumor weight=(length×width²)/2.

Log cell kill was also used as a measure of anti-tumor activity according to the following equation: log cell kill=T−C/(3.32×TVDT). In this equation: T=mean tumor weight at the end of treatment for the treated group; C=mean tumor weight at the end of treatment for the control group; and TVDT=median time (days) for control tumor weight to reach target size−median time (days) for control tumor weight to reach half the target size.

Therapeutic results were reported at the optimal dose, and results were not used if more than one death occurred in the treated group. A maximum tolerated dose (MTD), frequently equivalent to the optimal dose, was defined as a dose immediately below that causing unacceptable toxicity (i.e. more than one death) or, in the absence of any deaths, when accompanied by >20% loss of body weight. Statistical evaluations of in vivo efficacy of dasatinib (between-group comparisons) were performed using Gehan's generalized Wilcoxon test.³²

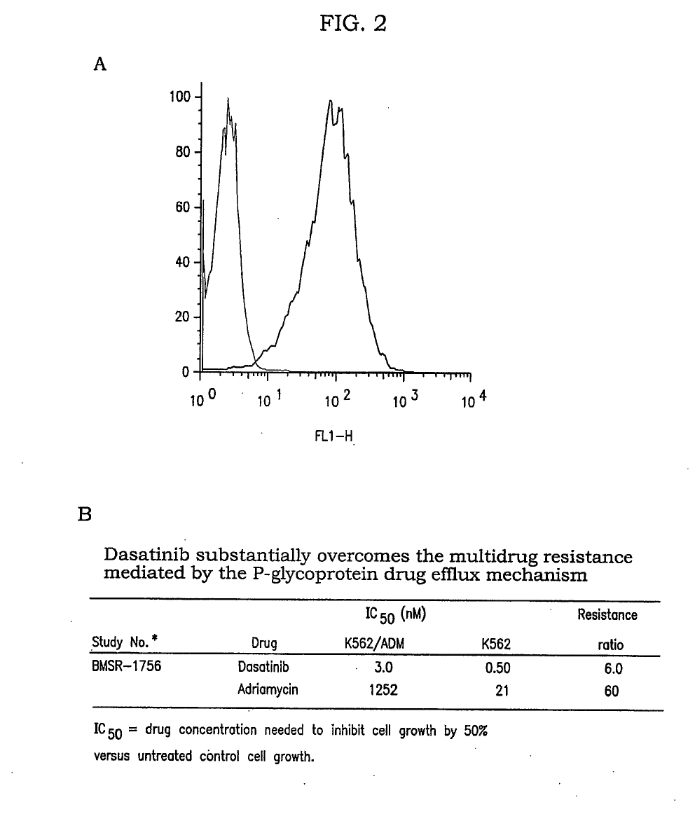

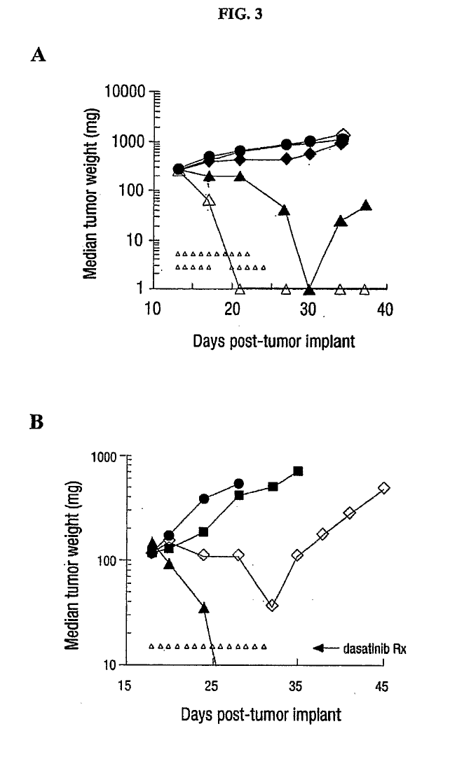

The results of these experiments demonstrated that K562/STI-571/R-derived tumor xenografts were as effectively induced in SCID mice as with the parental K562 cell line. Treatment with imatinib at 75 and 150 mg/kg three times daily had little or no impact on the tumors, which grew, like untreated controls (FIG. 2A). In contrast, treatment with dasatinib resulted in substantial reductions in tumor size in these mice at all doses, except for the lowest tested (5 mg/kg). However, it should be noted that this low dose of dasatinib was not without effect. Although the tumor regressed initially, sufficient cells probably remained for its return after treatment discontinuation. This somewhat resembles the results of treatment discontinuation in CML patients with varying degrees of residual disease.

It has been hypothesized that residual CML cells may at least in part arise from a pool of primitive CD34⁺ CD38⁻ cells with stem-cell properties, including the overexpression of MDR-1 drug transporter proteins. Accordingly, the anti-leukemic activity of dasatinib in the MDR-1-overexpressing K562/ADM leukemia model in vivo was evaluated. Dasatinib was administered orally once daily for 14 consecutive days to mice bearing SC K562/ADM leukemia. As tested, dasatinib completely eliminated tumors at 30 mg/kg and was active at 15 mg/kg, producing significant anti-leukemic activity (growth delay=16.7 days, P=0.0009). The dose of 5 mg/kg was inactive (FIG. 2B).

Overall, the results show that dasatinib can substantially overcome the Pgp-mediated MDR-1 resistance inherent in imatinib treated K562/ADM cells both in vitro and in vivo. In vitro, adriamycin suffers a 60-fold loss of potency in K562/ADM cells compared with K562 parent cells. The potency of dasatinib was reduced by 6-fold in K562/ADM, but with the IC₅₀ remaining at the low nM range (˜3 nM), effective exposure is expected to be still achievable in vivo in mice and in patients.

In addition, it has been proposed that primitive CD34⁺ CD38⁻ leukemic cells with stem-cell like properties may be the major source of residual disease and the reason for disease relapse upon treatment cessation. While multiple mechanisms are likely responsible for the persistence of the stem-like cells, one potential explanation is the expression of members of the ABC superfamily of transporter molecules, including Pgp, in primitive AML cells, and this may also be the case in CML cells.⁴⁷ The present finding of potent activity of dasatinib against Pgp-overexpressing CML cells suggests that dasatinib may have biologically meaningful activity against this cell population. The recent observation that dasatinib may have improved activity over imatinib in primary human primitive CML cells from patients is consistent with this observation.⁴⁸

These findings demonstrate that dasatinib is able to effectively overcome imatinib resistance of MDR-1 overexpression cell lines. The latter has important implications for leukemia patients who may be susceptible to the incidence of CNS complications subsequent to the administration of a protein tyrosine kinase inhibitor, such as imatinib, due to the significantly decreased sensitivity of MDR-1 overexpressing cells to imatinib. The findings also establish the utility of identifying patients who may be in need of dasatinib therapy, either alone or in combination with another compound, and/or an increased dose of dasatinib or an increased dosing frequency of dasatinib or a more aggressive dosing regimen, by comparing the expression level of MDR-1 in a patient sample relative to a standard or normal sample, wherein an elevated expression level is indicative that such dasatinib therapy may be warranted.

### Example 4

### Method of Determining Whether Dasatinib Confers Increased Survival to Mice with Intracranial CML In Vivo

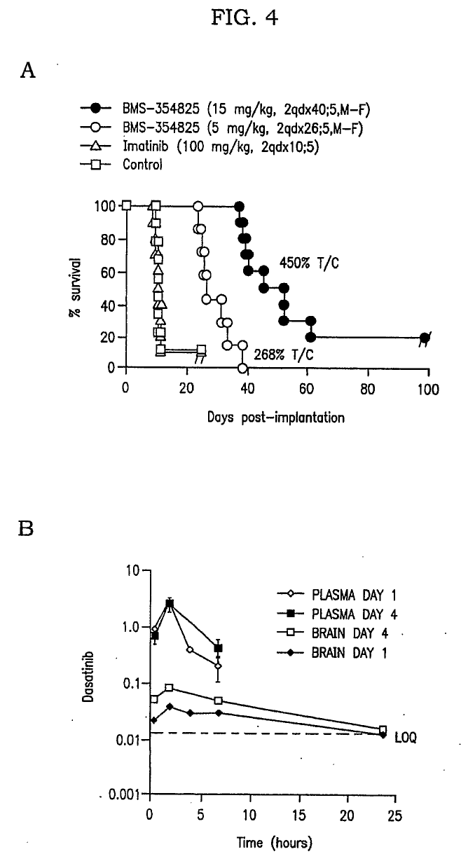

Extramedullary complications, particular in the CNS, are known risks of Ph+ALL or Ph+CML in lymphoid blast crisis. Recently, several reports indicated that imatinib has low efficacy in the CNS, possibly because of increased efflux of the agent from the CSF by p-glycoprotein.⁴² CML patients receiving treatment with imatinib alone remain at risk of developing CNS blast crisis, even when they are in complete or major cytogenetic response.^{55; 43; 44} Because dasatinib is 300-1000 fold more potent in killing Ph+CML cells and because it is effective in a nonclinical leukemia model with known PGP-mediated imatinib resistance (see above), it was proposed that dasatinib may be able to gain access into the CNS sufficiently to produce a therapeutically beneficial effect on Ph+CML and ALL with CNS involvement. To test this hypothesis, we evaluated the activity of dasatinib and imatinib in a head-to-head comparative study using a K562 intracranial CML tumor model. Both drugs were administered orally to mice inoculated intracranially (IC) with K562 cells using a BID×10 treatment schedule. Imatinib was administered at 100 mg/kg/adm, whereas dasatinib was administered at 5 mg/kg/adm or 15 mg/kg/adm. Dasatinib treatment significantly prolonged the survival of the leukemic mice, increasing lifespan (ILS) by 268% and 450% relative to imatibib which was effectively inactive (ILS=125%) (FIG. 4A).

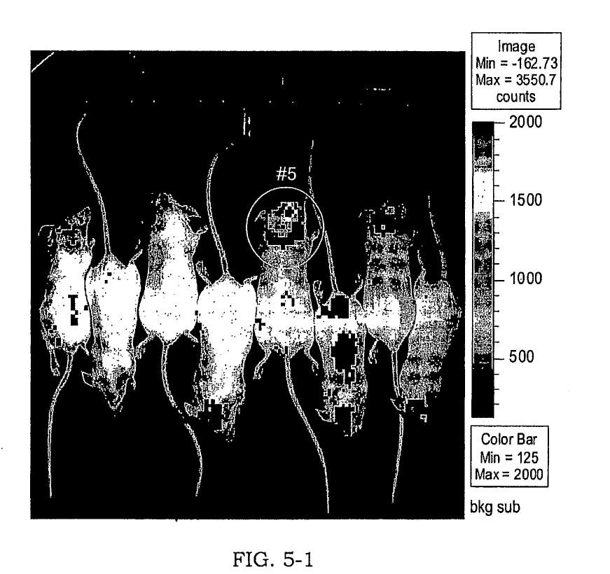

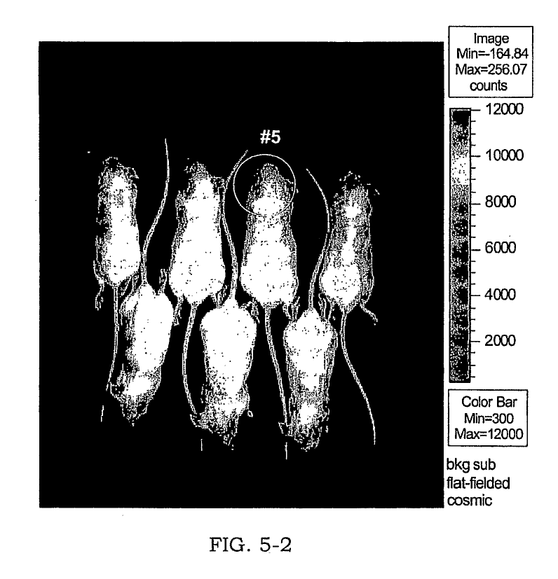

These positive results of the effects of dasatinib on the survival of leukemic mice have been subsequently reproduced in spectrophotometric studies in IC inoculated K562 in SCID mice using a bioluminescence imaging technique. As shown in FIG. 5, IC inoculated K562 SCID mice show bioluminescence in the absence of dastanib (see FIG. 5A), while virtually no bioluminescence was observed in mice treated with dasatinib despite increasing the spectrophotometer gain 6-fold (15 mg/kg, 2qdx14;6 μM-F,po) (see FIG. 5B).

### Example 5

### Method Used to Assess MDR-1 Expression Profile Using mRNA from Tissue and Cell Sources

Total RNA was purified using RNeasy system (Qiagen, CA, USA). Mixed Oligo-d(T)₁₅ primers were used to generate single-stranded cDNAs using the Superscript First-strand Synthesis kit (Invitrogen, CA, USA). Levels for MDR-1 and GAPDH transcripts were analyzed using an Applied Biosystems 7900HT Sequence Detection System. Mixed primer/probe sets for each transcript (MDR-1, catalog #HS00184491_ml; GAPDH, catalog #4326317E) were obtained from Applied Biosystems and used according to the manufacturer's instructions.

Expression levels of MDR-1 transcripts were normalized to endogenous GAPDH transcripts. Comparisons were made between samples by ΔΔCt comparative analysis using manufacturer's software (Applied Biosystems). Briefly, ΔCT=(MDR CT)−(GAPDH CT); ΔΔCT=(ΔCT^{Probe1}−ΔCT^{Probe2}); and Fold change=2^{ΔΔC}.

### Example 6

### Additional Method of Assessing the Expression Profile of the MDR-1 Using mRNA from Tissue and Cell Sources

Total RNA from tissues may be isolated using the TriZol protocol (Invitrogen) and quantified by determining its absorbance at 260 nM. An assessment of the 18s and 28s ribosomal RNA bands can be made by denaturing gel electrophoresis to determine RNA integrity.

The specific sequence to be measured can be aligned with related genes found in GenBank to identity regions of significant sequence divergence to maximize primer and probe specificity. Gene-specific primers and probes may be designed using the ABI primer express software to amplify small amplicons (150 base pairs or less) to maximize the likelihood that the primers function at 100% efficiency. All primer/probe sequences are then searched against Public Genbank databases to ensure target specificity. Primers and probes were obtained from ABI.

For MDR-1 (NM_{—}000927 and gi|2353263), the following, non-limiting, primer probe sequences may be used:

| | |
|---|---|
| Forward Primer | |
| 5′- gctctggccttctggtatgggacc-3′ | (SEQ ID NO: 1) |
| Reverse Primer | |
| 5′- gaaaatacagtgagtacttgtcc-3′ | (SEQ ID NO: 2) |
| TaqMan Probe | |
| 5′- ccttggtcctctcaggggaatattc-3′ | (SEQ ID NO: 3) |

### DNA Contamination

To access the level of contaminating genomic DNA in the RNA, the RNA can be divided into 2 aliquots and one half to be treated with Rnase-free Dnase (Invitrogen). Samples from both the Dnase-treated and non-treated are then subjected to reverse transcription reactions with (RT+) and without (RT−) the presence of reverse transcriptase. TaqMan assays are carried out with gene-specific primers (see above) and the contribution of genomic DNA to the signal detected then evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+ RNA. If not the RNA was not used in actual experiments.

### Reverse Transcription Reaction and Sequence Detection

100 ng of Dnase-treated total RNA is annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP is added to the reaction and the tube is incubated at 37° C. for 30 min. The sample is then heated to 90° C. for 5 min to denature enzyme.

Quantitative sequence detection is carried out on an ABI PRISM 7700 by adding to the reverse transcribed reaction 2.5 μM forward and reverse primers, 2.0 μl of the TaqMan probe, 500 μM of each dNTP, buffer and 5U AmpliTaq Gold™. The PCR reaction is then held at 94° C. for 12 min, followed by 40 cycles of 94° C. for 15 sec and 60° C. for 30 sec.

### Data Handling

The threshold cycle (Ct) of a standard gene is used as the baseline of expression and all other tissues are standardized relative to the same. The relative abundance of MDR-1 in the sample relative to the standard gene is calculated according to the difference in Ct value between the standard and the MDR-1 sample and using it as the exponent in 2^{(ΔCt)}

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, Genbank Accession numbers, SWISS-PROT Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

What is claimed is:

1. A method for determining the responsiveness of an individual with a BCR-ABL associated disorder to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, comprising:
screening a biological sample from said individual for the presence of MDR-1 overexpression; wherein the presence of said overexpression is indicative of the individual being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, therapy.

2. The method of claim 1 wherein the individual has not previously been treated with a kinase inhibitor.

3. The method of claim 1 wherein the individual has been previously treated with a kinase inhibitor and has developed at least partial resistance to the kinase inhibitor.

4. The method of claim 3 wherein the kinase inhibitor is N-(2-chloro-6-methylphenyl)-2-[[6-[4-(2-hydroxyethyl)-1 piperazinyl]-2-methyl-4-pyrimidinyl]amino]-5-thiazolecarboxamide or a pharmaceutically acceptable salt, solvate, or hydrate thereof.

5. The method of claim 6 wherein the kinase inhibitor is imatinib, AMN107, PD180970, CGP76030, AP23464, SKI 606, or AZD0530.

6. The method of claim 1 wherein the BCR-ABL-associated disorder is leukemia, breast cancer, prostate cancer, lung cancer, colon cancer, melanoma, or solid tumors.

7. The method of claim 6 wherein the leukemia is chronic myeloid leukemia (CML), Ph+ALL, AML, imatinib-resistant CML, imatinib-intolerant CML, accelerated CML, or lymphoid blast phase CML.

8. A method of treating an individual suffering from a BCR-ABL-associated disorder comprising:
determining whether a biological sample obtained from the individual has MDR-1 overexpression, wherein the presence of said overexpression is indicative of the patient being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, therapy; and
administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual.

9. The method of claim 8 wherein the thiazolecarboxamide, or a pharmaceutically acceptable salt, solvate, or hydrate thereof, is administered at a higher dosage or dosing frequency if it is determined that the biological sample has MDR-1 overexpression

10. The method of claim 9, wherein the thiazolecarboxamide or pharmaceutically acceptable salt, hydrate, or solvate thereof is administered at a dosage of greater than 70 mg twice daily.

11. The method of claim 10, wherein the thiazolecarboxamide, or a pharmaceutically acceptable salt, solvate, or hydrate thereof, is administered in combination with a second therapy to treat the protein tyrosine kinase associated disorder in the individual.

12. The method of claim 11, wherein the second therapy is a tubulin stabilizing agent, a farnysyl transferase inhibitor, a BCR-ABL T315I inhibitor, a second protein tyrosine kinase inhibitor, or a combination thereof.

13. A method of diminishing the incidence of CNS complications for CML patients, comprising the step of determining whether a biological sample obtained from the individual has MDR-1 overexpression, wherein the presence of overexpressed MDR-1 warrants administration of a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual.

14. The method of claim 13 wherein the thiazolecarboxamide, or a pharmaceutically acceptable salt, solvate, or hydrate thereof, is administered at a higher dosage or dosing frequency if it is determined that the biological sample has MDR-1 overexpression

15. The method of claim 14, wherein the thiazolecarboxamide or pharmaceutically acceptable salt, hydrate, or solvate thereof is administered at a dosage of greater than 70 mg twice daily.

16. The method of claim 13, wherein the thiazolecarboxamide, or a pharmaceutically acceptable salt, solvate, or hydrate thereof, is administered in combination with a second therapy to treat the protein tyrosine kinase associated disorder in the individual.

17. The method of claim 16, wherein the second therapy is a tubulin stabilizing agent, a farnysyl transferase inhibitor, a BCR-ABL T3151 inhibitor, a second protein tyrosine kinase inhibitor, or a combination thereof.

18. A kit for use in determining treatment strategy for an individual with a BCR-ABL-associated disorder, comprising
a means for determining whether a biological sample obtained from said individual has MDR-1 overexpression; and optionally
instructions for use and interpretation of the kit results.

19. The kit of claim 18, wherein said kit comprises said instructions and wherein said treatment strategy comprises administration of a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate or solvate thereof.

20. A kit for use in treating an individual with a BCR-ABL associated disorder, comprising:
a means for determining whether a biological sample obtained from said individual has MDR-1 overexpression;
a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate or solvate thereof; and
instructions for use of said kit.
